(19) United States
(12) Patent Application Publication
(10) Pub. No.: US 2026/0201340 A1
(43) Pub. Date: Jul. 16, 2026

(54) MEDIUM COMPOSITION FOR PREPARATION OF CANCER ORGANOID

(71) Applicant: ORGANOIDSCIENCES LTD., Seongnam-si, Gyeonggi-do (KR)

(72) Inventors: Sarang KIM, Seongnam-si (KR); Bo Eun LEE, Seongnam-si (KR); Woo Kyeom YANG, Seongnam-si (KR); Dong Hyeon KIM, Seongnam-si (KR); Da Bin PYEON, Seongnam-si (KR); Si Na KIM, Seongnam-si (KR)

(73) Assignee: ORGANOIDSCIENCES LTD., Seongnam-si, Gyeonggi-do (KR)

(21) Appl. No.: 19/134,020

(22) PCT Filed: Nov. 22, 2023
(86) PCT No.: PCT/KR2023/018875

§ 371 Date: May 29, 2025

(30) Foreign Application Priority Data
Nov. 30, 2022 (KR) 10-2022-0165265

(51) Int. Cl. C12N 5/09; G01N 33/50

(52) U.S. Cl. CPC C12N 5/0693; G01N 33/5011; C12N 2500/05; C12N 2500/30; C12N 2500/35; C12N 2500/38; C12N 2500/90; C12N 2501/11; C12N 2501/727

(57) ABSTRACT

Provided is a medium composition for preparing a cancer organoid and a method for preparing a cancer organoid, comprising a step of culturing using the medium composition; and a cancer organoid prepared by the preparation method. A medium for preparing a cancer organoid according to the present invention can promote proliferation of the cancer organoid and maintain stemness of the cancer organoid. The medium can be used to prepare cancer organoids of uniform quality in large quantities within a short period of time, and the cancer organoids prepared with the medium can also be used to directly observe effects on cancer cell proliferation/metastasis and anticancer drug resistance, and can be used in the development of a drug screening platform to check patient-specific anticancer drug resistance in advance. Observation of gene expression regulation according to drug treatment can contribute to the study of molecular mechanisms for the purpose of cancer treatment.

### TECHNICAL FIELD

This application claims the benefit of priority based on Korean Patent Application No. 10-2022-0165265, filed on Nov. 30, 2022, and the entire disclosure of the Korean patent application is incorporated herein as a part of this specification.

The present invention relates to a medium composition for preparing cancer organoids. The present invention also relates to a method for preparing cancer organoids comprising a step of culturing using the medium composition, and to cancer organoids prepared by the method.

### BACKGROUND ART

Stem cells possess unique characteristics of differentiation ability (multi-potency) and self-renewal, and accordingly, research has been continuously conducted to utilize them in various applications such as treatment of incurable diseases, disease modeling, transplantation of tissues or organs, and drug efficacy evaluation. In particular, it has been revealed that when stem cells are cultured in an appropriate three-dimensional in vitro environment, cell clusters with structural and tissue characteristics similar to in vivo organs are formed, and such cell clusters are referred to as organoids.

Organoid production technology is expected to be utilized in applications such as efficacy evaluation or screening of various therapeutic agents, and transplantation into tissues or organs. Organoids may be more effective in testing the safety and efficacy of new drugs compared to two-dimensional cell tissues, and are considered to be applicable for improving conditions through transplantation into damaged or underdeveloped organs. Accordingly, organoid-related research is becoming increasingly active from the perspective of regenerative medicine, and organoids are anticipated to be widely applicable across various fields.

However, since the technologies for preparing and culturing organoids are still in the early stages of research, various studies are required to establish preparing and culturing methods, such as determining the combination or ratio of medium additives. Although medium compositions for proliferative liver organoid differentiation and methods for producing liver organoids using the same, as well as methods for producing brain organoids, have been developed, there remains a need to develop medium compositions tailored to each organ and methods for producing organoids using such compositions.

### Technical Problem

The inventors have conducted various studies to efficiently prepare cancer organoids with uniform quality. As a result, a novel component which is capable of maximizing the promotion of proliferation and the maintenance of stemness for cancer organoids was identified among various media components, and the present invention was completed by experimentally demonstrating its effect.

### Technical Solution

Each description and embodiment disclosed in the present invention is also applicable to other descriptions and embodiments. That is, all combinations of the various elements disclosed herein fall within the scope of the present invention. In addition, it cannot be considered that the scope of the present invention is limited by the specific descriptions disclosed below.

In addition, it should be understood that terms not specifically defined in this specification have meanings commonly used in the technical field to which the present invention pertains. Also, unless otherwise defined by context, the singular includes the plural, and the plural includes the singular.

One aspect of the present invention provides a medium composition for preparing cancer organoids, comprising dorsomorphin.

The medium composition of the present invention promotes the proliferation of cancer organoids, enabling the formation of a large number of cancer organoids within a short period of time. The formed cancer organoids maintain stemness, thereby ensuring uniform quality. Accordingly, the medium composition of the present invention can be usefully applied to the production of cancer organoids for various fields such as regenerative therapeutics and drug screening.

As used in the present specification, the term “organoid” refers to a three-dimensional (3D) cell aggregate. It is defined as a miniaturized and simplified version of an organ produced through an artificial culturing process, without being collected, obtained, or harvested from animals, etc. Organoids have the advantages of long-term culturing, cryopreservation, and ease of manipulation and observation. In addition, since immortalization is not required, the intrinsic characteristics of the cells can be maintained, and the organoids serve as experimental models that enable the study of physiological phenomena at a higher level than individual cells by reproducing the hierarchical and histological structures of cells as observed only in vivo.

As used in the present specification, the term “cancer organoid” refers to an organoid that possesses a structure, cellular composition, and function identical or similar to those of cancer in vivo, and may be produced by culturing cells derived from cancer tissue or cancer cells. The term “cancer organoid” may be used interchangeably with “tumor organoid” or “tumoroid”.

The cancer may be, but not limited to, colorectal cancer, pancreatic cancer, lung cancer, biliary tract cancer, gastric cancer, liver cancer, colon cancer, small intestine cancer, brain cancer, bone cancer, melanoma, breast cancer, sclerosing adenosis, uterine cancer, cervical cancer, head and neck cancer, esophageal cancer, thyroid cancer, parathyroid cancer, kidney cancer, sarcoma, prostate cancer, urethral cancer, bladder cancer, hematologic cancer, leukemia, lymphoma, fibroadenoma, and the like.

In the present invention, the cancer organoid may be cultured for three or more passages, five or more passages, or ten or more passages.

As used in the present specification, the term “passage” refers to a cycle of replacing the culture vessel or subdividing the cell population in order to continuously culture cells or organoids in a healthy state over an extended period of time by successively propagating the cell lineage. Increasing the number of passages enables the acquisition of a larger number of organoids. One passage refers to a single replacement of the culture vessel or a single subdivision of the cell population, whereas twenty passages refer to twenty replacements of the culture vessel or twenty subdivisions of the cell population. One passage may be carried out over a period of 1 to 10 days. In the present specification, the term “passage” may be used interchangeably with the term “generation”.

As used in the present specification, the term “dorsomorphin” refers to a compound named 6-[4-(2-piperidin-1-ylethoxy)phenyl]-3-pyridin-4-ylpyrazolo[1,5-a]pyrimidine.

In the present invention, when cancer organoids are produced using a medium comprising dorsomorphin, not only is the proliferation of cancer organoids promoted, resulting in a significantly increased number of cancer organoids obtained, but also the proliferation rate of the cancer organoids is increased, enabling the acquisition of a desired amount of cancer organoids within a short period of time. In addition, the cancer organoids produced according to the present invention can maintain stemness at an excellent level even when the number of passages increases or they are cultured for a long period, thereby allowing for more accurate evaluation of the efficacy of anticancer agents or screening of anticancer agents using such cancer organoids. The content of dorsomorphin in the medium may be easily selected by those skilled in the art depending on the type of cancer, cells, or organoids.

As used in the present specification, the term “preparation” may be used interchangeably with “culturing” or “proliferation”. Accordingly, the medium composition for preparing cancer organoids may be referred to as a medium composition for culturing cancer organoids, a medium composition for the formation of cancer organoids, or a medium composition for expanding cancer organoids. In this context, “formation” refers to the process of forming cancer organoids from cancer-derived cells and exhibiting organ- or tissue-like characteristics so as to perform the inherent functions of cancer. In addition, “expansion” refers to increasing the number of formed cancer organoids to obtain a sufficient quantity for the intended purpose.

As used in the present specification, the term “medium” refers to a mixture of nutrients that enables the growth, survival, expansion, or differentiation of cells or organoids in vitro, and includes all appropriate conventional media used in the art. Depending on the type of cell or organoid, the type of medium and culturing conditions may be selected within the technical level of those skilled in the art. The medium may be a basal medium for cell culture containing a carbon source, nitrogen source, and trace elements, and specific examples include, but are not limited to, DMEM (Dulbecco's Modified Eagle's Medium), MEM (Minimal Essential Medium), BME (Basal Medium Eagle), RPMI1640, F-10, F-12, a-MEM (a-Minimal Essential Medium), GMEM (Glasgow's Minimal Essential Medium), IMDM (Iscove's Modified Dulbecco's Medium), DMEM/F12 (Dulbecco's Modified Eagle Medium/Nutrient Mixture F-12), or Advanced DMEM/F12.

Organoids have conventionally been prepared using two types of culture media. For example, an organoid formation medium has been used to form organoids from cells or tissues collected or isolated from a subject and to promote their growth in size, followed by the use of an organoid expansion medium, after removing the formation medium, to increase the number of organoids. When the organoid formation medium and the expansion medium are composed of different components, the preparation procedure becomes cumbersome and complicated, and has disadvantages in that it requires considerable time and expense.

In addition, due to their inherent characteristics, organoids exhibit significantly different preparation outcomes depending on the culture conditions. Examples of culture conditions include culture duration, culture temperature, culture medium, and culture matrix, all of which are important factors in determining the properties of the organoids. Among them, the medium conditions play the most critical role in enabling organoids to grow with characteristics similar to the desired tissue or organ by regulating various signaling pathways in stem cells.

In the present invention, dorsomorphin was identified as a medium component capable of yielding a sufficient number of cancer organoids with structure and function identical or highly similar to tissue in vivo, and in particular, with increased proliferative capacity and stemness, enabling their application in drug screening or efficacy evaluation. Furthermore, it was confirmed that when using a medium containing dorsomorphin, cancer organoid formation, growth, and expansion can be successfully induced, and the rate of these processes can be increased, even without using two or more types of culture media, but with only a single type of culture medium.

In addition, the medium composition for preparing cancer organoids according to the present invention may further comprise NRG1.

As used in the present specification, the term “NRG1” refers to the Neuregulin 1 protein encoded by the NRG1 gene. Information regarding its protein sequence and the like may be found in publicly available databases (e.g., NCBI Reference Sequence: NP_001153467, NP_001153468, NP_001153471, NP_001153473, NP_001153474). The NRG1 may be comprised in the medium composition of the present invention at a concentration of 1 to 100 nM, for example, at a concentration of 50 nM, but is not limited thereto.

In addition, the medium composition for preparing cancer organoids according to the present invention may further comprise an antioxidant. The antioxidant is preferably at least one selected from the group consisting of valproic acid, boric acid, resveratrol, edaravone and ascorbic acid, and more preferably at least one selected from the group consisting of boric acid, resveratrol, edaravone and ascorbic acid.

The sources for obtaining dorsomorphin, NRG1, and the antioxidant are not particularly limited. For example, commercially available substances may be used, or they may be prepared using genetic (or protein) recombinant technology according to methods known in the art.

In addition, the medium composition of the present invention may be a medium composition for promoting the preparation of cancer organoids. The medium composition of the present invention may also be a medium composition for increasing the preparation rate of cancer organoids. Furthermore, the medium composition of the present invention may be a medium composition for promoting the formation, growth, or expansion of cancer organoids.

Another aspect of the present invention provides a method for preparing cancer organoids, comprising a step of culturing cancer cells isolated from a subject using the above-described medium composition.

In the method for preparing cancer organoids according to the present invention, each term shall have the same meaning as described in the medium composition for preparing cancer organoids, unless specifically stated otherwise.

As used in the present specification, the term “subject” includes individuals who have never developed cancer, individuals who may potentially develop cancer, individuals who have developed cancer, and individuals who have been cured after developing cancer, and may include, without limitation, humans or any non-human animals. The non-human animals may be vertebrates such as primates, dogs, cattle, horses, pigs, and rodents such as mice, rats, hamsters, and guinea pigs. In this specification, the term “subject” may be used interchangeably with “individual” or “patient”.

The cancer cells isolated from the subject may include stem cells.

In addition, the isolated cancer cells may be obtained through a cancer tissue dissection process and an enzymatic digestion process. Specifically, the dissection may include both physical dissection and mechanical dissection and may be performed using general tissue dissection methods known in the art. In addition, the enzymatic digestion may be performed under general enzymatic digestion conditions known in the art, and may, for example, be carried out using one or more enzymes selected from the group consisting of dispase II, deoxyribonuclease (DNase) I and collagenase II.

In addition, culture conditions such as culture duration, culture temperature, and culture matrix may be applied under conditions commonly used in the art for culturing organoids, and one of ordinary skill in the art will be able to use methods suitable for the objectives of the present invention.

Another aspect of the present invention provides a cancer organoid prepared by the method for preparing cancer organoids described above.

In the cancer organoid according to the present invention, unless specifically stated otherwise, each term has the same meaning as described in the medium composition for preparing cancer organoids.

The cancer organoid according to the present invention maintains stemness and has histological composition and function highly similar to those of human cancer, and thus can be usefully applied for screening or efficacy evaluation of anticancer drugs or therapeutic agents targeting tumors. In particular, when the cancer organoid is prepared using patient-derived cells or tissues, it can be used to predict patient-specific anticancer drug resistance, non-responsiveness, or refractoriness. Since most cancer patients have a short remaining lifespan, it is necessary to quickly identify and administer the most effective anticancer drug for the individual. To this end, personalized medicine and precision medicine are being developed, and the cancer organoid derived from the cancer tissue of a specific patient according to the present invention can be effectively applied. In addition, the cancer organoid can contribute to the study of molecular mechanisms for cancer treatment by observing the regulation of gene expression in response to drug treatment.

Another aspect of the present invention provides a method for evaluating the efficacy of an anticancer agent, comprising a step of treating the above-described cancer organoid with the anticancer agent.

Cancer organoids derived from patient's cancer tissues are highly similar to actual cancer tissues and have the advantage that patient-specific genotypes and phenotypes are well preserved in vitro, and thus can be effectively applied to evaluate efficacy for overcoming resistance to anticancer agents or therapeutic agents targeting tumors.

As used in the present specification, the term “anticancer agent resistance” may also be referred to as “anticancer agent refractoriness” or “anticancer agent non-responsiveness,” and refers to a case where there is no therapeutic effect from the beginning of treatment with an anticancer agent in a cancer patient, or the effect diminishes or is lost during continuous treatment, or the response to treatment does not persist over a long period.

Another aspect of the present invention provides a method for screening anticancer agents, comprising a step of treating the above-described cancer organoid with a candidate anticancer agent.

Cancer organoids derived from patient cancer tissues are highly similar to actual cancer tissues and have the advantage that patient-specific genotypes and phenotypes are well preserved in vitro. Accordingly, by treating the cancer organoids with a candidate anticancer agent and measuring the proliferative capacity, metastatic potential and anticancer agent resistance of the cancer organoids, the cancer organoids can be usefully applied in a platform for anticancer agent screening.

As used in the present specification, the term “candidate substance” refers to a substance expected to be capable of treating cancer. Specifically, any substance that is expected to inhibit or improve the growth, migration, or metastasis of cancer cells, or increase the apoptosis of cancer cells, may be used without limitation, and includes all therapeutic candidate substances such as compounds, genes, or proteins.

In the method for evaluating anticancer agent efficacy or the method for screening anticancer agents according to the present invention, each term shall have the same meaning as described in the medium composition for preparing cancer organoids, unless specifically stated otherwise.

The medium for preparing cancer organoids according to the present invention can promote the proliferation of cancer organoids and maintain their stemness. Accordingly, the medium can be used to produce a large quantity of cancer organoids with uniform quality in a short period of time. It is also possible to directly observe the effects on cancer cell proliferation/metastasis and anticancer agent resistance using the cancer organoids produced with the medium. Furthermore, it can be applied to the development of drug screening platforms and used to predict patient-specific anticancer agent resistance in advance. In addition, by observing gene expression regulation in response to drug treatment, it can contribute to molecular mechanism studies for the purpose of cancer therapy.

### BRIEF DESCRIPTION OF FIGURES

FIG. 1 is an image showing the appearance of cancer organoids prepared using media containing different main components. “All” refers to a medium containing dorsomorphin, NRG1, and resveratrol; “-Dorsomorphin and NRG1” refers to a medium excluding dorsomorphin and NRG1 but containing resveratrol; “-Dorsomorphin and Resveratrol” refers to a medium excluding dorsomorphin and resveratrol but containing NRG1; and “—Dorsomorphin” refers to a medium excluding dorsomorphin but containing NRG1 and resveratrol. “P0+0d” is a photograph taken on day 0 after culturing, “P0+9d” is a photograph taken on day 9 after culturing, and “P1+9d” is a photograph taken on day 9 after the first passage from the start of culturing (the same applies to FIG. 2).

FIG. 2 is a graph showing the growth rates of cancer organoids prepared using media with different components.

FIG. 3 is an image showing the appearance of cancer organoids prepared using media containing dorsomorphin, NRG1 and antioxidants as main components, where the type of antioxidant varies between media. “+Valproic acid” refers to a medium containing valproic acid as the antioxidant; “+Boric acid” refers to a medium containing boric acid; “+Resveratrol” refers to a medium containing resveratrol; “+Edaravone” refers to a medium containing edaravone; and “+Ascorbic acid” refers to a medium containing ascorbic acid. “P6+0d” is a photograph taken on day 0 after the sixth passage from the start of culturing; “P6+3d” is a photograph taken on day 3 after the sixth passage; and “P6+10d” is a photograph taken on day 10 after the sixth passage (the same applies to FIG. 4).

FIG. 4 is a graph showing the growth rates of cancer organoids prepared using media containing dorsomorphin, NRG1 and antioxidants as main components, with different types of antioxidants.

### EXAMPLES

Hereinafter, the present invention will be described in further detail through examples. These examples are provided for more specific illustration of the present invention and are not intended to limit the scope of the present invention. In this specification, unless otherwise specifically defined in context, the singular shall include the plural, and the plural shall include the singular.

### Example 1. Culturing of Cancer Organoids Depending on Medium Composition

Advanced DMEM/F12 was used as the basal medium for culturing cancer organoids, and dorsomorphin, NRG1 (Neuregulin 1), and an antioxidant were selectively added. Specifically, the experimental groups were classified as follows:
(1) “All”: containing dorsomorphin, NRG1, and resveratrol
(2) “-Dorsomorphin and NRG1”: excluding dorsomorphin and NRG1, containing resveratrol
(3) “-Dorsomorphin and Resveratrol”: excluding dorsomorphin and resveratrol, containing NRG1
(4) “-Dorsomorphin”: excluding dorsomorphin, containing NRG1 and resveratrol

In the media of (1) to (4) above, dorsomorphin was used at 250 nM, NRG1 at 50 nM, and resveratrol at 100 nM.

Colorectal cancer tissue obtained via endoscopic biopsy or surgical resection from a patient was washed three or more times using a washing solution. The washed colorectal cancer tissue was treated with Collagenase II at 37° C. for 2 hours. Then, a 0.1% BSA (bovine serum albumin) in DPBS was further added to the tissue, and pipetting was performed to isolate crypts containing colorectal cancer stem cells. The isolated crypts were passed through a 70 μm cell strainer and then centrifuged. Subsequently, the obtained crypts were mixed at a 1:1 ratio with an extracellular matrix (collagen or matrigel) and cultured in a three-dimensional form. The above media (1) to (5) were used to perform 3D culturing for 4 to 9 days per passage. The area of colorectal cancer organoids formed in each medium was measured, and the growth rate was analyzed using the following formula. The results are shown in Table 1 below.

$$Cancer organoid growth rate ( \% ) = ( Area of cancer organoids at P 0 + 0 d , P 0 + 9 d , or P 1 + 9 d after culturing ) / ( Area of cancer organoids at P 0 + 0 d after culturing ) \times 100$$

TABLE 1

| Culture | | −Dorsomorphin | −Dorsomorphin | |
|---|---|---|---|---|
| Day | All | and NRG1 | and Resveratrol | −Dorsomorphin |
| P0 + 0 d | 100 (%) | 100 (%) | 100 (%) | 100 (%) |
| P0 + 9 d | 180 (%) | 135 (%) | 216 (%) | 222 (%) |
| P1 + 9 d | 294 (%) | 121 (%) | 60 (%) | 58 (%) |

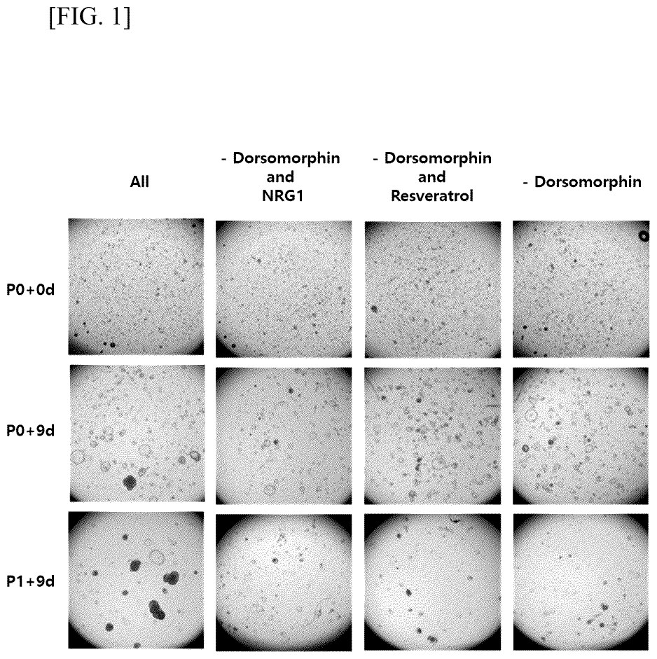

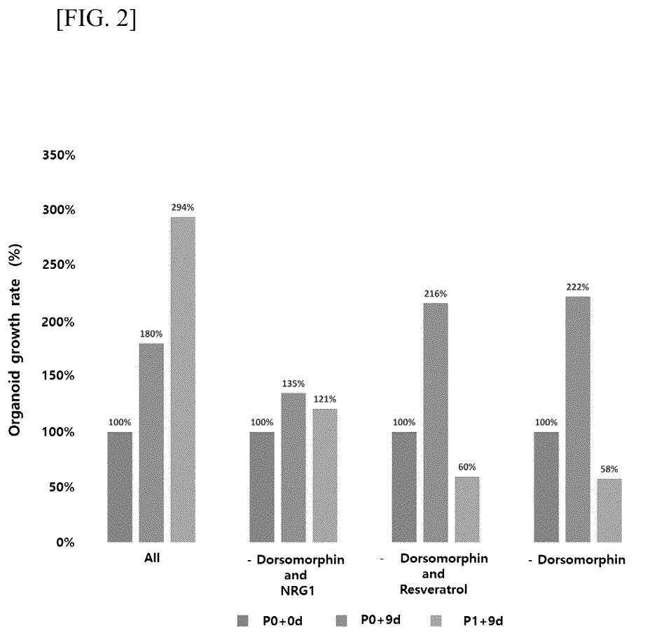

As shown in Table 1, FIG. 1 and FIG. 2, when the medium containing dorsomorphin, NRG1 and resveratrol (“All”) was used, the growth rate of the organoids increased as the culture progressed. In contrast, in media excluding dorsomorphin and NRG1 but containing resveratrol (“-Dorsomorphin and NRG1”), excluding dorsomorphin and resveratrol but containing NRG1 (“-Dorsomorphin and Resveratrol”), and excluding dorsomorphin (“-Dorsomorphin”), a decrease in organoid growth rate was observed when compared at the same time points.

In particular, when the medium lacking dorsomorphin (“-Dorsomorphin”) was used, the growth rate decreased sharply by approximately fourfold as passaging progressed.

These results suggest that in order to promote the proliferation of cancer organoids and secure a greater number of organoids, dorsomorphin is required as an essential component in the medium used for culturing cancer organoids.

### Example 2. Culturing of Cancer Organoids Depending on Antioxidant Type

To compare the effects of different types of antioxidants on the culture of cancer organoids, all other conditions except for the antioxidant type were kept constant, and the medium compositions were prepared by varying only the type of antioxidant.

Specifically, cancer cells were continuously passaged from “P0” to “P4” to obtain a sufficient number of cells for the experiment. From passage “P5”, Advanced DMEM/F12 was used as the basal medium, with dorsomorphin and NRG1 comprised as key components, and the antioxidants valproic acid, boric acid, resveratrol, edaravone, or ascorbic acid were selectively added.

The experimental groups were classified as follows:
(1) “+Valproic acid”: containing dorsomorphin, NRG1, and valproic acid
(2) “+Boric acid”: containing dorsomorphin, NRG1, and boric acid
(3) “+Resveratrol”: containing dorsomorphin, NRG1, and resveratrol
(4) “+Edaravone”: containing dorsomorphin, NRG1, and edaravone
(5) “+Ascorbic acid”: containing dorsomorphin, NRG1, and ascorbic acid

Colorectal cancer organoids were cultured using the media (1) to (5) in the same manner as in Example 1. Dorsomorphin was used at a concentration of 250 nM, NRG1 at 50 nM, and the antioxidants were used at the following concentrations: resveratrol, 100 nM; boric acid, 1 mM; edaravone, 50 μM; valproic acid, 1 mM; and ascorbic acid, 284 μM.

Subsequently, the area of the colorectal cancer organoids prepared with each medium composition was measured, and the growth rate was analyzed using the following formula. The results for passage 6 (“P6”), after the sixth passage of culturing, are shown in Table 2 below.

$$Cancer organoid growth rate ( \% ) = ( Area of cancer organoids at P 6 + 0 d , P 6 + 3 d , or P 6 + 10 d after culturing ) / ( Area of cancer organoids at P 6 + 0 d after culturing ) \times 100$$

TABLE 2

| Culture | +Valproic | +Boric | | | +Ascorbic |
|---|---|---|---|---|---|
| Day | Acid | Acid | +Resveratrol | +Edaravone | acid |
| P6 + 0 d | 100 (%) | 100 (%) | 100 (%) | 100 (%) | 100 (%) |
| P6 + 3 d | 105 (%) | 128 (%) | 112 (%) | 131 (%) | 113 (%) |
| P6 + 10 d | 262 (%) | 409 (%) | 458 (%) | 456 (%) | 529 (%) |

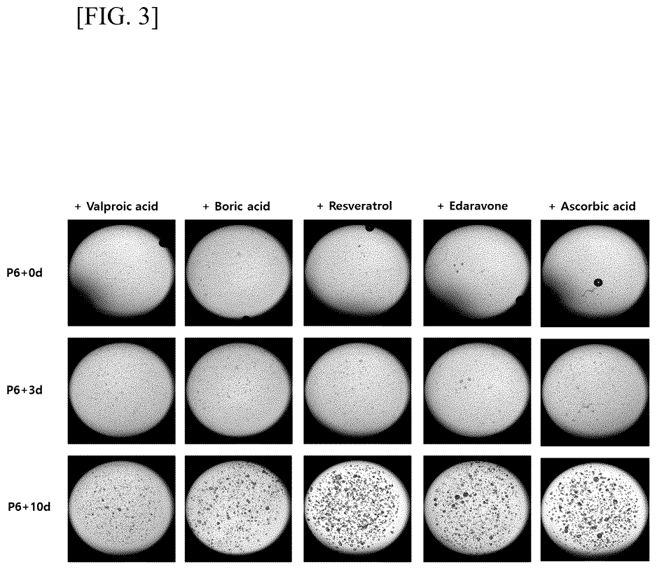

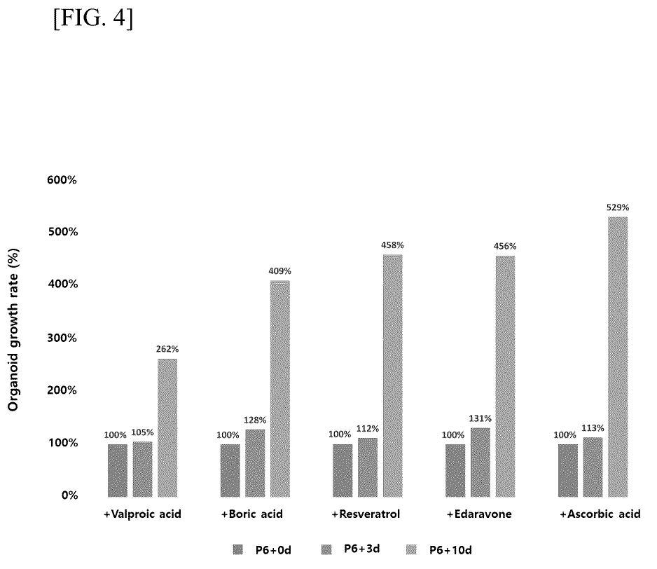

As shown in Table 2, FIG. 3, and FIG. 4, differences in organoid growth rates were observed depending on the type of antioxidant used. Specifically, at “P6+3d”, the growth rates of the organoids were similar across conditions; however, as the culture period progressed, the differences in growth rate became more pronounced. At “P6+10d”, compared to “P6+0d”, the following increases in growth rate were observed: approximately 2.6-fold for the +Valproic Acid medium, approximately 4.1-fold for the +Boric Acid medium, approximately 4.6-fold for the +Resveratrol medium, approximately 4.6-fold for the +Edaravone medium, and approximately 5.3-fold for the +Ascorbic acid medium.

Based on the results of Examples 1 and 2, it was confirmed that dorsomorphin is required as an essential component in the medium composition for preparing cancer organoids, in order to secure a large number of cancer organoids and maintain their stemness during long-term culture. Therefore, in order to promote the proliferation of cancer organoids and significantly reduce the time required to obtain the desired quantity of cancer organoids, dorsomorphin is required in the medium composition for preparing cancer organoids.

What is claimed is:

1. A medium composition for preparing cancer organoids, comprising dorsomorphin.

2. The medium composition according to claim 1, wherein the cancer is at least one selected from the group consisting of colorectal cancer, pancreatic cancer, lung cancer, biliary tract cancer and gastric cancer.

3. The medium composition according to claim 1, wherein the composition further comprises a basal medium.

4. The medium composition according to claim 3, wherein the basal medium is DMEM, MEM, BME, RPMI1640, F-10, F-12, α-MEM, GMEM, IMDM, DMEM/F12 or Advanced DMEM/F12.

5. The medium composition according to claim 1, wherein the composition further comprises at least one selected from the group consisting of NRG1 and an antioxidant.

6. The medium composition according to claim 5, wherein the antioxidant is at least one selected from the group consisting of valproic acid, boric acid, resveratrol, edaravone and ascorbic acid.

7. The medium composition according to claim 1, wherein the composition is for promoting the preparation of cancer organoids.

8. A method for preparing cancer organoids, comprising a step of culturing cancer cells isolated from a subject using the medium composition of claim 1.

9. A cancer organoid prepared by the method of claim 8.

10. A method for evaluating the efficacy of an anticancer agent, comprising a step of treating the cancer organoid of claim 9 with the anticancer agent.

11. A method for screening an anticancer agent, comprising a step of treating the cancer organoid of claim 9 with a candidate anticancer agent.
